(19) United States
(12) Patent Application Publication
(10) Pub. No.: US 2019/0262004 A1
(43) Pub. Date: Aug. 29, 2019

(54) COUPLING DEVICE FOR INFLATION AND DEFLATION ASSEMBLY

(71) Applicant: Easynotes Ltd., Kfar Truman (IL)

(72) Inventors: Nir Altman, Kfar Etzion (IL); Izhak Fabian, Kfar Truman (IL)

(73) Assignee: Easynotes Ltd., Kfar Truman (IL)

(21) Appl. No.: 15/906,023

(22) Filed: Feb. 27, 2018

(51) Int. Cl. A61B 17/12

(52) U.S. Cl. CPC A61B 17/12136; A61M 25/10181; A61B 17/12099; A61B 17/12031

(57) ABSTRACT

An inflation and deflation assembly includes an interface member including a proximal connector portion and distal resilient fingers cantilevered from an intermediate body portion distal of the proximal connector portion. A coupling device is formed with a lumen that has a proximal lumen portion and a distal lumen portion and a constriction portion intermediate the proximal lumen portion and the distal lumen portion. In a coupling orientation, the interface member is introduced into the coupling device and the resilient fingers bend radially inwards upon abutting and passing through the constriction portion.

### FIELD OF THE INVENTION

The present invention generally relates to fluid delivery devices, such as for introducing fluid for inflating balloons placed in body lumens and for extracting fluids from the balloons for their deflation, e.g., for inflating and deflating balloons used to obstruct or reduce flow of gastric contents across the pyloric valve.

### BACKGROUND OF THE INVENTION

U.S. Pat. No. 9,125,660 describes an obstruction device (also called pyloric plug) for obstructing or reducing flow through a body lumen, in particular for obstructing or reducing flow of gastric contents across the pyloric valve (pylorus). The device is particularly useful in a transoral gastrointestinal procedure, but may be used in other laparoscopic, endoscopic, or natural orifice procedures in other body lumens. The plug is designed to be fully operative over a long time, and it can be removed and re-implanted.

The plug includes two balloons, one proximal and the other distal, mounted on a shaft. The proximal obstruction balloon is arranged to fit in the stomach, whereas the distal obstruction balloon is arranged to fit in the duodenum. When inflated, both balloons expand towards the pylorus from opposite sides on the pylorus, thus fixing the plug in place.

The plug is particularly useful to stop the flow of stomach contents to the proximal gut which includes the duodenum and the initial part of the jejunum. Such a need arises, for example, after creating an alternative path of flow through a gastro-jejunum anastomosis which bypasses the proximal gut. There could be other cases when this need arises, such as after surgery in the duodenum area or in the pancreas or bile outputs to the duodenum. Another indication could be the need to operate endoscopically on the stomach with an inflated stomach. In this case, the plug keeps the inflating air in the stomach and it does not bloat the intestine.

### SUMMARY OF THE INVENTION

The present invention seeks to provide a coupling device for ensuring secure coupling of a catheter and needle through which fluid is required to flow for inflating and deflating items, such as balloons placed in body lumens, as is described more in detail hereinbelow. The device is particularly useful for inflating and deflating balloons located at proximal and distal sides of the pylorus, but may be used for other balloons and other inflatable devices as well.

One of the advantages of the inflation and deflation assembly of the invention over the prior art is that the coupling device allows for two-stage coupling of the catheter and needle. In the first stage, an interface member, to which the catheter is attached, is introduced into the coupling device with a relatively low insertion force. At this point, the force required to retract and withdraw the interface member and catheter is also a relatively low retraction force. In the second stage, the needle is introduced through the catheter and interface member into the coupling device and by doing so significantly increases the retraction force required to retract and withdraw the interface member and catheter. In this manner, the catheter and needle are securely held in the coupling device for the entire duration of inflation or deflation. The needle is also introduced with hardly any insertion force, but the presence of the needle prevents resilient fingers of the interface member from bending inwards. Any attempt to retract or pull out the catheter from the coupling device causes the resilient fingers to press outwards against the inner perimeter of the coupling device, and significantly increases the force required to retract the catheter from the coupling device. As long as the needle is inserted, the catheter will not pull out from the coupling device. The needle must be removed in order to decouple the catheter from the coupling device.

There is thus provided in accordance with an embodiment of the present invention an inflation and deflation assembly including an interface member including a proximal connector portion and distal resilient fingers cantilevered from an intermediate body portion distal of the proximal connector portion, and a coupling device formed with a lumen that has a proximal lumen portion and a distal lumen portion and a constriction portion intermediate the proximal lumen portion and the distal lumen portion. In a coupling orientation, the interface member is introduced into the coupling device and the resilient fingers bend radially inwards upon abutting and passing through the constriction portion.

### BRIEF DESCRIPTION OF THE DRAWINGS

The present invention will be understood and appreciated more fully from the following detailed description taken in conjunction with the drawings in which:

FIG. 1 is a simplified illustration of an inflation and deflation assembly, constructed and operative in accordance with a non-limiting embodiment of the present invention;

FIG. 2 is a simplified illustration of an interface member used in the inflation and deflation assembly, constructed and operative in accordance with a non-limiting embodiment of the present invention;

FIG. 3 is a simplified sectional illustration of a coupling device used in the inflation and deflation assembly, constructed and operative in accordance with a non-limiting embodiment of the present invention;

FIG. 4 is a simplified cutaway illustration of the interface member of the inflation and deflation assembly introduced into the coupling device; and

FIG. 5 is a simplified cutaway illustration of the needle of the inflation and deflation assembly introduced through the interface member into the coupling device.

### DETAILED DESCRIPTION OF EMBODIMENTS

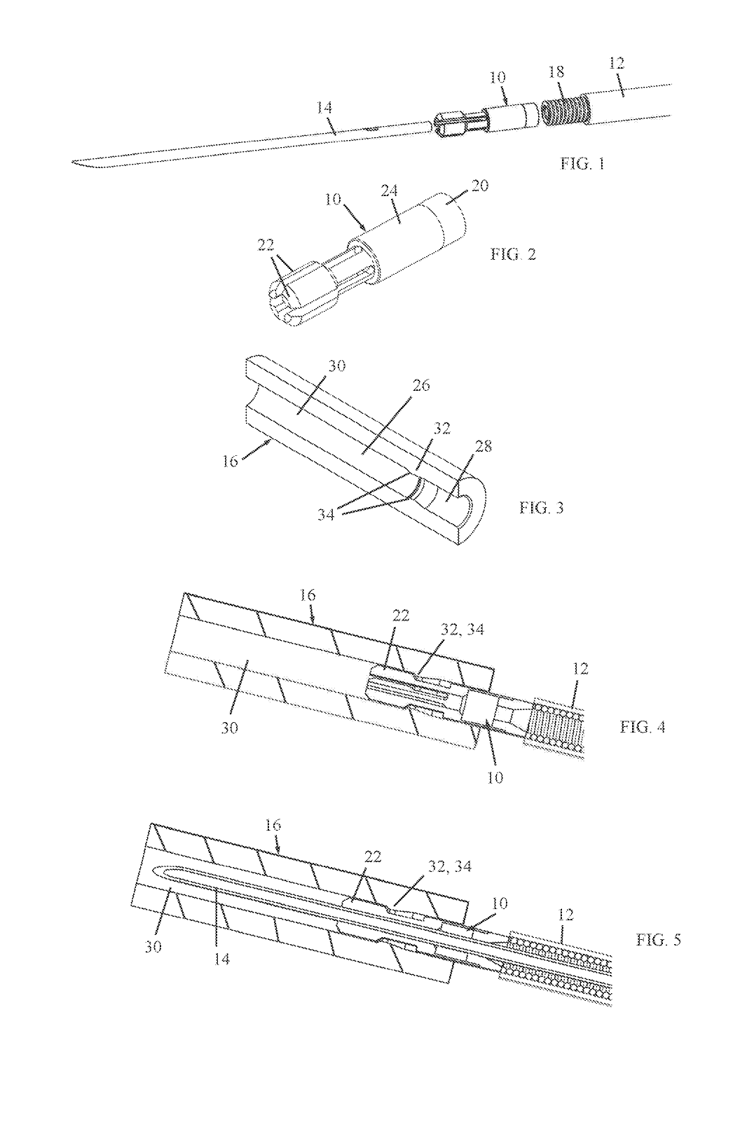

Reference is now made to FIG. 1, which illustrates an inflation and deflation assembly, constructed and operative in accordance with a non-limiting embodiment of the present invention.

The inflation and deflation assembly includes an interface member 10, a catheter 12, a needle 14 and a coupling device 16 (FIG. 3). Catheter 12 may be any kind of tube, flexible or rigid, made of any suitable, medically safe material. In the non-limiting illustrated embodiment, catheter 12 includes coils 18 for bendability and good torque transmission.

Reference is now made to FIG. 2, which illustrates the interface member 10. Interface member 10 includes a proximal connector portion 20 which couples with a distal portion of catheter 12. For example, the coils 18 snugly fit into the inner diameter of the proximal connector portion 20 or may be joined to portion 20, such as by welding, bonding or any other method. Interface member 10 includes distal resilient fingers 22 cantilevered from an intermediate body portion 24 distal of the proximal connector portion 20.

As seen in FIG. 3, coupling device 16 is formed with a lumen 26 that has a proximal lumen portion 28 and a distal lumen portion 30 and a constriction portion 32 intermediate the proximal lumen portion 28 and distal lumen portion 30. The constriction portion 32 has a diameter smaller than the rest of lumen 26. The constriction portion 32 includes an annular bump 34 which protrudes radially inwards.

Coupling device 16, in use, may be positioned in a balloon which is to be inflated or deflated.

One of the advantages of the inflation and deflation assembly over the prior art is that the coupling device 16 allows for two-stage coupling of the catheter 12 and needle 14, and there is no need for rotational alignment of the parts being coupled (since they are aligned at any arbitrary rotational angle), as is now explained with reference to FIGS. 4 and 5.

As seen in FIG. 4, in the first stage, interface member 10, to which the catheter 12 is attached, is introduced into the coupling device 16 with a relatively low insertion force. The insertion force is low because the resilient fingers 22 easily yield and bend radially inwards upon abutting and passing through the constriction portion 32, or in the illustrated embodiment, passing through bump 34. The resilient fingers 22 expand radially outwards back to their initial configuration after passing through bump 34. At this point, the force required to retract and withdraw interface member 10 and catheter 12 is also a relatively low retraction force.

In the second stage, shown in FIG. 5, needle 14 is introduced through catheter 12 and interface member 10 into coupling device 16. Needle 14 is introduced with hardly any insertion force, but the presence of needle 14 prevents resilient fingers 22 from bending inwards. Any attempt to retract catheter 12 from coupling device 16 causes resilient fingers 22 to press outwards against the inner perimeter of coupling device 16, and significantly increases the force required to retract catheter 12 from coupling device 16. As long as needle 14 is inserted, catheter 12 will not pull out from coupling device 16, and catheter 12 and needle 14 are securely held in coupling device 16 for the entire duration of inflation or deflation. Needle 14 must be removed in order to decouple catheter 12 from coupling device 16.

What is claimed is:

1. An inflation and deflation assembly comprising:
an interface member comprising a proximal connector portion and distal resilient fingers cantilevered from an intermediate body portion distal of said proximal connector portion, and
a coupling device formed with a lumen that has a proximal lumen portion and a distal lumen portion and a constriction portion intermediate said proximal lumen portion and said distal lumen portion,
wherein in a coupling orientation, said interface member is introduced into said coupling device and said resilient fingers bend radially inwards upon abutting and passing through said constriction portion.

2. The inflation and deflation assembly according to claim 1, wherein said constriction portion comprises an annular bump which protrudes radially inwards.

3. The inflation and deflation assembly according to claim 1, further comprising a needle introduced through said interface member into said coupling device.

4. An inflation and deflation assembly comprising:
an interface member, a catheter, a needle and a coupling device,
wherein said interface member comprises a proximal connector portion, which couples with a distal portion of said catheter, and distal resilient fingers cantilevered from an intermediate body portion distal of said proximal connector portion, and
wherein said coupling device is formed with a lumen that has a proximal lumen portion and a distal lumen portion and a constriction portion intermediate said proximal lumen portion and said distal lumen portion.

5. The inflation and deflation assembly according to claim 4, wherein said constriction portion comprises an annular bump which protrudes radially inwards.

6. The inflation and deflation assembly according to claim 4, wherein in a coupling orientation, said interface member is introduced into said coupling device and said resilient fingers bend radially inwards upon abutting and passing through said constriction portion.

7. The inflation and deflation assembly according to claim 4, wherein in a coupling orientation, said needle is introduced through said interface member into said coupling device.
